(19) United States
(12) Patent Application Publication
(10) Pub. No.: US 2024/0017247 A1
(43) Pub. Date: Jan. 18, 2024

(54) Catalyst, Method for Producing Catalyst, and Method for Producing alpha,beta-Unsaturated Aldehyde, alpha,beta-Unsaturated Carboxylic Acid and alpha,beta-Unsaturated Carboxylic Acid Ester

(71) Applicant: Mitsubishi Chemical Corporation, Tokyo (JP)

(72) Inventors: Yu Kurihara, Tokyo (JP); Kensuke Nishiki, Tokyo (JP)

(73) Assignee: Mitsubishi Chemical Corporation, Tokyo (JP)

(21) Appl. No.: 18/371,240

(22) Filed: Sep. 21, 2023

Related U.S. Application Data
(63) Continuation of application No. PCT/JP2022/012989, filed on Mar. 22, 2022.

(30) Foreign Application Priority Data
Mar. 24, 2021 (JP) 2021-049747

(51) Int. Cl. B01J 23/887; B01J 23/00; B01J 37/04; B01J 37/08; B01J 6/00

(52) U.S. Cl. CPC B01J 23/8876; B01J 23/002; B01J 37/04; B01J 37/08; B01J 6/001

(57) ABSTRACT

An object of the present invention is to provide a catalyst with high yields of target products such as an α,β-unsaturated aldehyde and an α,β-unsaturated carboxylic acid. Problems are solved by a catalyst containing at least molybdenum and having a COD (chemical oxygen demand) of the catalyst of greater than 300 ppm and less than 11,000 ppm.

### CROSS-REFERENCE TO RELATED APPLICATION

This is a continuation of International Application PCT/JP2022/012989, filed on Mar. 22, 2022, and designated the U.S., and claims priority from Japanese Patent Application 2021-049747 which was filed on Mar. 24, 2021, the entire contents of which are incorporated herein by reference.

### TECHNICAL FIELD

The present invention relates to a catalyst, a method of producing the catalyst, and a method of producing an α,β-unsaturated aldehyde, an α,β-unsaturated carboxylic acid, and an α,β-unsaturated carboxylic acid ester.

### BACKGROUND ART

In production processes of organic compounds such as α,β-unsaturated aldehydes and α,β-unsaturated carboxylic acids, catalysts containing molybdenum are often used. Catalytic performance of these catalysts has been known to vary depending on their physical properties, and many investigations have been conducted to control these physical properties.

Patent Document 1 describes use of a catalyst containing molybdenum, bismuth, iron, cobalt, and lanthanide elements with a controlled ratio of Fe²⁺/(Fe²⁺+Fe³⁺), in production of an unsaturated aldehyde using an olefin and/or an alcohol as raw materials.

Patent Document 2 describes a catalyst for production of unsaturated aldehydes and/or unsaturated carboxylic acids, composed of a composite oxide containing molybdenum, bismuth, and iron, in which calcination of the catalyst in the presence of a reducing substance and controlling a reduced mass percentage upon the calcination, thereby provide a catalyst with an excellent mechanical strength.

Patent Document 3 describes a heteropolyacid-based catalyst for production of methacrylic acid, containing a water-soluble heteropolyacid and water-hardly soluble heteropolyacid salt, in which controlling degrees of reduction of the water-soluble heteropolyacid and water-hardly soluble heteropolyacid salt in the catalyst, thereby provides a catalyst with high productivity of methacrylic acid.

### RELATED ART DOCUMENTS

### Patent Documents

[Patent Document 1] Japanese Unexamined Patent Application Publication No. 2014-161775
[Patent Document 2] Japanese Unexamined Patent Application Publication No. 2009-274034
[Patent Document 3] Japanese Unexamined Patent Application Publication No. 2008-284439

### SUMMARY OF THE INVENTION

### Problem to be Solved by the Invention

However, the catalysts described in Patent Document 1 to 3 do not always have a sufficient yield of an α,β-unsaturated aldehyde and a sufficient yield of an α,β-unsaturated carboxylic acid. Therefore, from the viewpoint of further improving catalytic performance, catalytic properties of the catalysts are required to be controlled.

The present invention was made in view of the aforementioned circumstances, and an object of the present invention is to provide a catalyst with high yield of target products such as an α,β-unsaturated aldehyde and an α,β-unsaturated carboxylic acid.

### Means for Solving the Problems

The present inventors have conducted diligent investigations in order to achieve the aforementioned object. As a result, the present inventors have found that in a molybdenum-containing catalyst, an index referred to a COD (chemical oxygen demand), which indicates an oxidation-reduction condition of the catalyst, has a significant effect on its catalytic performance, and that controlling the COD within a certain range thereby provides a catalyst with high yield of a target product, and thus have completed the present invention.

Namely, the present invention includes as described below:
[1]: A catalyst comprising at least molybdenum, wherein a COD (chemical oxygen demand) of the catalyst is greater than 300 ppm and less than 11,000 ppm.
[2]: The catalyst according to [1], wherein a value obtained by dividing the COD value (ppm) by a value of specific surface area S (m²/g) of the catalyst (COD/S), is greater than 43 μg/m² and 3600 μg/m² or less.
[3]: The catalyst according to [1] or [2], for use in production of an α,β-unsaturated aldehyde and/or an α,β-unsaturated carboxylic acid from an alkene, an alcohol, or an ether.
[4]: The catalyst according to any one of [1] to [3], having a composition represented by the following formula (1):

Moₐ₁Bi_{b1}Fe_{c1}M_{d1}Xₑ₁Y_{f1}Si_{g1}(NH₄)ₕ₁Oᵢ₁ (1)
wherein in formula (1) above, Mo, Bi, Fe, Si, NH₄, and O each represent molybdenum, bismuth, iron, silicon, an ammonium root, and oxygen; M represents at least one element selected from the group consisting of cobalt and nickel; X represents at least one element selected from the group consisting of zinc, chromium, lead, manganese, calcium, magnesium, niobium, silver, barium, tin, tantalum, tungsten, antimony, phosphorus, boron, sulfur, selenium, tellurium, cerium and titanium; Y represents at least one element selected from the group consisting of lithium, sodium, potassium, rubidium, cesium, and thallium; a1, b1, c1, d1, e1, f1, g1, h1, and it each represent a molar ratio of each component, and when a1=12, b1=0.01 to 3, c1=0 to 8, d1=0 to 12, e1=0 to 8, f1=0.001 to 2, g1=0 to 20, h1=0 to 30, and it is a molar ratio of oxygen required to satisfy a valence of the each component.
[5]: The catalyst according to claim [3] or [4], wherein the COD is greater than 300 ppm and 2,000 ppm or less.
[6]: The catalyst according to any one of [3] to [5], wherein the COD is from 400 to 1,500 ppm.
[7]: The catalyst according to any one of [3] to [6], wherein the COD/S is from 45 to 500 μg/m².
[8]: The catalyst according to [1] or [2], for use in production of an α,β-unsaturated carboxylic acid from an α,β-unsaturated aldehyde.
[9]: The catalyst according to any one of [1], [2], and [8], having a composition represented by the following formula (2):

Pₐ₂Mo_{b2}V_{c2}Cu_{d2}Aₑ₂E_{f2}G_{g2}(NH₄)ₕ₂Oᵢ₂ (2)
wherein in formula (2) above, P, Mo, V, Cu, NH₄, and O each represent phosphorus, molybdenum, vanadium, copper, an ammonium root, and oxygen; A represents at least one element selected from the group consisting of antimony, bismuth, arsenic, germanium, zirconium, tellurium, silver, selenium, silicon, tungsten and boron; E represents at least one element selected from the group consisting of iron, zinc, chromium, magnesium, calcium, strontium, tantalum, cobalt, nickel, manganese, barium, titanium, tin, lead, niobium, indium, sulfur, palladium, gallium, cerium, and lanthanum; G represents at least one element selected from the group consisting of lithium, sodium, rubidium, potassium, cesium and thallium; and a2, b2, c2, d2, e2, f2, g2, h2 and i2 each represent a molar ratio of each component, and when b2=12, a2=0.5 to 3, c2=0.01 to 3, d2=0.01 to 2, e2=0 to 3, f2=0 to 3, g2=0 to 5, h2=0 to 30, and i2 is a molar ratio of oxygen required to satisfy a valence of the each component.
[10]: The catalyst of [8] or [9], wherein the COD is 2,500 ppm or greater and less than 11,000 ppm.
[11]: The catalyst according to any one of [8] to [10], wherein the COD is from 2,600 to 10,000 ppm.
[12]: The catalyst according to any one of [8] to [11], wherein the COD/S is from 100 to 3,000 μg/m².
[13]: A method of producing a catalyst comprising at least molybdenum, wherein the method comprises the following steps (i) to (v):
(i) mixing at least a molybdenum raw material with a solvent to obtain a slurry (liquid A);
(ii) stirring the liquid A at a temperature of 1 to 30° C. lower than the boiling point of the solvent for 20 to 90 minutes to obtain a slurry (liquid B);
(iii) stirring the liquid B at a temperature of 2° C. or higher than the temperature in the step (ii) for 10 minutes to 10 hours to obtain a slurry (liquid C);
(iv) drying the liquid C to obtain a dried product; and (v) calcining the dried product to obtain a catalyst.
[14]: The method of producing the catalyst according to [13], wherein 50% by mass or more of the total solvent is water in the step (i).
[15]: The method of producing the catalyst according to [13] or [14], wherein the temperature in the step (iii) is 1 to 20° C. higher than the boiling point of the solvent.
[16]: The method of producing a catalyst according to any one of to [15], comprising stirring the liquid B for minutes to 10 hours to obtain the liquid C in the step (iii).
[17]: The method of producing a catalyst according to any one of to [16], comprising calcining the dried product under oxygen-containing gas distribution in the step (v).
[18]: The method of producing a catalyst according to any one of to [17], comprising producing a catalyst used when an α,β-unsaturated aldehyde and/or an α,β-unsaturated carboxylic acid are/is produced from an alkene, an alcohol, or an ether.
[19]: The method of producing a catalyst according to any one of to [17], comprising producing a catalyst used when an α,β-unsaturated carboxylic acid is produced from an α,β-unsaturated aldehyde.
[20]: A method of producing an α,β-unsaturated aldehyde and/or an α,β-unsaturated carboxylic acid, comprising producing the α,β-unsaturated aldehyde and/or the α,β-unsaturated carboxylic acid from an alkene, an alcohol or an ether by using the catalyst according to any one of [1] to [7].
[21]: A method of producing an α,β-unsaturated aldehyde and/or an α,β-unsaturated carboxylic acid, comprising producing the α,β-unsaturated aldehyde and/or the α,β-unsaturated carboxylic acid from an alkene, an alcohol or an ether by using a catalyst produced by the production method according to any one of to [18].
[22]: A method of producing an α,β-unsaturated carboxylic acid, comprising producing the α,β-unsaturated carboxylic acid from an α,β-unsaturated aldehyde by using the catalyst according to any one of [1], [2], and [8] to [12].
[23]: A method of producing an α,β-unsaturated carboxylic acid, comprising producing the α,β-unsaturated carboxylic acid from an α,β-unsaturated aldehyde by using a catalyst produced by the production method according to to [17] and [19].
[24]: A method of producing an α,β-unsaturated carboxylic acid, comprising producing the α,β-unsaturated carboxylic acid from an α,β-unsaturated aldehyde produced by the production method according to or [21].
[25]: A method of producing an α,β-unsaturated carboxylic acid ester, comprising producing the α,β-unsaturated carboxylic acid ester from an α,β-unsaturated carboxylic acid produced by the production method according to any one of [20] to [24].

### Effects of the Invention

According to the present invention, a catalyst with high yield of target products can be provided.

### MODE FOR CARRYING OUT THE INVENTION

Embodiments according to the present invention will be described below, however, the present invention is not limited as described below. Moreover, the phrase “XX or more and YY or less” or “XX to YY,” which denotes a numerical value range, refers to a numerical value range including lower limit and upper limit, which are the endpoints, unless otherwise specified. When numerical ranges are described stepwisely, the upper limit and lower limit of each numerical range can be arbitrarily combined.

[Catalyst]

The catalyst according to the present invention is a catalyst containing at least molybdenum, and a COD (chemical oxygen demand) of the catalyst is greater than 300 ppm and less than 11,000 ppm. Using such a catalyst enables production of a target product from a raw material in high yield.

The catalyst according to the present invention is preferably an oxidation catalyst from the viewpoint of a target product yield, and is more preferably a catalyst used for producing an α,β-unsaturated aldehyde and/or an α,β-unsaturated carboxylic acid. Specifically, it is preferably a catalyst used for producing an α,β-unsaturated aldehyde and/or an α,β-unsaturated carboxylic acid from an alkene, an alcohol or an ether, or a catalyst used for producing an α,β-unsaturated carboxylic acid from an α,β-unsaturated aldehyde. Incidentally, the phrase “producing an α,β-unsaturated aldehyde and/or an α,β-unsaturated carboxylic acid” denotes that either of the α,β-unsaturated aldehyde and α,β-unsaturated carboxylic acid may be produced or both thereof may be produced.

(Composition of Catalyst)

The catalyst according to the present invention contains at least molybdenum, and preferably has the composition represented by the following formula (1) or (2) from the viewpoint of a target product yield. When the catalyst according to the present invention is a catalyst used when an α,β-unsaturated aldehyde and/or an α,β-unsaturated carboxylic acid are/is produced from an alkene, alcohol, or ether, the composition represented by the following formula (1), provides an α,β-unsaturated aldehyde and/or an α,β-unsaturated carboxylic acid in high yield. Moreover, when the catalyst according to the present invention is a catalyst used when an α,β-unsaturated carboxylic acid is produced from an α,β-unsaturated aldehyde, the composition represented by the following formula (2) provides an α,β-unsaturated carboxylic acid in high yield. Note, however, the catalyst component may contain a small amount of element not listed in the following formula (1) or (2).

Moₐ₁Bi_{b1}Fe_{c1}M_{d1}Xₑ₁Y_{f1}Si_{g1}(NH₄)ₕ₁Oᵢ₁ (1)

In formula (1) above, Mo, Bi, Fe, Si, NH₄, and O each represent molybdenum, bismuth, iron, silicon, an ammonium root, and oxygen; M represents at least one element selected from the group consisting of cobalt and nickel; X represents at least one element selected from the group consisting of zinc, chromium, lead, manganese, calcium, magnesium, niobium, silver, barium, tin, tantalum, tungsten, antimony, phosphorus, boron, sulfur, selenium, tellurium, cerium and titanium; Y represents at least one element selected from the group consisting of lithium, sodium, potassium, rubidium, cesium, and thallium; a1, b1, c1, d1, e1, f1, g1, h1, and it each represent a molar ratio of each component, and when a1=12, b1=0.01 to 3, c1=0 to 8, d1=0 to 12, e1=0 to 8, f1=0.001 to 2, g1=0 to 20, h1=0 to 30, and it is a molar ratio of oxygen required to satisfy a valence of the each component.

Pₐ₂Mo_{b2}V_{c2}Cu_{d2}Aₑ₂E_{f2}G_{g2}(NH₄)ₕ₂Oᵢ₂ (2)

In formula (2) above, P, Mo, V, Cu, NH₄, and O each represent phosphorus, molybdenum, vanadium, copper, ammonium root, and oxygen; A represents at least one element selected from the group consisting of antimony, bismuth, arsenic, germanium, zirconium, tellurium, silver, selenium, silicon, tungsten, and boron; E represents at least one element selected from the group consisting of iron, zinc, chromium, magnesium, calcium, strontium, tantalum, cobalt, nickel, manganese, barium, titanium, tin, lead, niobium, indium, sulfur, palladium, gallium, cerium, and lanthanum; G represents at least one element selected from the group consisting of lithium, sodium, rubidium, potassium, cesium, and thallium; a2, b2, c2, d2, e2, f2, g2, h2 and i2 each represent a molar ratio of each component, and when b2=12, a2=0.5 to 3, c2=0.01 to 3, d2=0.01 to 2, e2=0 to 3, f2=0 to 3, g2=0 to 5, h2=0 to 30, and i2 is a molar ratio of oxygen required to satisfy a valence of the each component.

It is noted that a molar ratio of each component shall be a value obtained by analyzing a catalyst dissolved in ammonia water by ICP atomic emission spectrometry. A molar ratio of ammonium root is also a value obtained by analyzing the catalyst by the Kjeldahl method.

When the catalyst according to the present invention has the elemental composition represented by formula (1) above, from the viewpoint of yields of α,β-unsaturated aldehyde and/or α,β-unsaturated carboxylic acid, when a1=12, the lower limit of b1 is preferably 0.03 or more, and more preferably 0.05 or more. The upper limit of b1 is also preferably 2 or less, and more preferably 1 or less. The lower limit of c1 is preferably 0.01 or more, more preferably or more, and further preferably 1 or more. The upper limit of c1 is also preferably 5 or less, and more preferably 3 or less. The lower limit of d1 is preferably 0.01 or more, more preferably 0.1 or more, further preferably 1 or more, and particularly preferably 3 or more. The upper limit of d1 is also preferably 10 or less, and more preferably 9 or less. The lower limit of e1 is preferably 0.1 or more, more preferably 0.2 or more, and further preferably 0.5 or more. The upper limit of e1 is also preferably 6 or less, and more preferably 4 or less. The lower limit of f1 is preferably or more, and more preferably 0.1 or more. The upper limit of f1 is also preferably 1.5 or less, and more preferably 1 or less. The lower limit of g1 may be 1 or more, and may be 5 or more. The upper limit of g1 is also preferably 15 or less, and more preferably 10 or less. The upper limit of h1 is preferably 20 or less, and more preferably 10 or less.

When the catalyst according to the present invention has the elemental composition represented by formula (2) above, from the viewpoint of the yield of α,β-unsaturated carboxylic acid, when b2=12, the lower limit of a2 is preferably 0.8 or more, and more preferably 1 or more. The upper limit of a2 is also preferably 2.5 or less, and more preferably 2 or less. The lower limit of c2 is preferably 0.1 or more and more preferably 0.2 or more. The upper limit of c2 is also preferably 2.5 or less, and more preferably 2 or less. The lower limit of d2 is preferably 0.05 or more, and more preferably 0.1 or more. The upper limit of d2 is also preferably 1 or less, and more preferably 0.5 or less. The lower limit of e2 may be 0.01 or more, and may be 0.1 or more. The upper limit of e2 is preferably 2.5 or less, and more preferably 2 or less. The lower limit of f2 may be 0.01 or more, and may be 0.03 or more. The upper limit of f2 is also preferably 2.5 or less, and more preferably 2 or less. The lower limit of g2 is preferably 0.1 or more, and may be 0.5 or more. The upper limit of g2 is also preferably 4 or less, and more preferably 3 or less. The upper limit of h2 is preferably 20 or less, and more preferably 10 or less.

The catalyst according to the present invention may also have a support for supporting catalytic active components. The supports are not particularly limited, and include silica, alumina, silica-alumina, magnesia, titania, silicon carbide, and the like. Of these, preferred is silica as a support in order to prevent the support itself from being reacted upon its use. Note, however, when the support is used for the catalyst in the present invention, catalysts including the support are regarded as the catalysts of the present invention.

(COD of Catalyst)

The COD of the catalyst represents a weight of oxygen molecules necessary for complete oxidation of unit weight of catalyst. When 1 μg of oxygen molecules is required to completely oxidize 1 g of the catalyst, a COD value is 1 ppm. Here, the unit of ppm represents μg/g.

The COD of the catalyst according to the present invention is greater than 300 ppm and less than 11,000 ppm. This allows production of target products in high yield. The reason is not clear but is presumed as follows. An active site of a catalyst used for production of organic compounds such as an α,β-unsaturated aldehyde and an α,β-unsaturated carboxylic acid, can take two types of states, such as an oxidation state and a reduction state. Then, the target products are produced through an oxidation-reduction cycle in which the active site changes between the oxidation state and reduction state. Therefore, in order for such an oxidation-reduction cycle to turn, the active site is required to be stable in both the oxidation state and the reduction state. Here, the COD of the catalyst is an index of an abundance ratio of the oxidation state and the reduction state as the entire catalyst, not limited to a specific element. When the COD of the catalyst is small, the abundance ratio of an oxidation state is large, indicating that the oxidation state is relatively stable. When the COD of the catalyst is large, on the other hand, the abundance ratio of a reduction state is large, indicating that the reduction state is relatively stable. The COD of the catalyst being greater than 300 ppm and less than 11,000 ppm, can be regarded that both an oxidation state and reduction state are stable. Thus, the oxidation-reduction cycle of the catalyst can easily turn, which is thereby considered to improve yields of target products.

The lower limit of COD of the catalyst is preferably 400 ppm or more, more preferably 450 ppm or more, further preferably 500 ppm or more, and particularly preferably 550 ppm or more. The upper limit of COD of the catalyst is preferably 10,000 ppm or less, more preferably 9,000 ppm or less, further preferably 8,000 ppm or less, and particularly preferably 7,400 ppm or less.

A preferred range of COD of the catalyst depends on an elemental composition of the catalyst and its use. When the catalyst is used in a reaction that requires a large number of moles of oxygen in order to react with one mole of a raw material substrate, an oxidation state is preferably more stable so that a reduction state facilitates return to the oxidation state, because many active sites in the reduction state are generated during the reaction. In other words, the COD preferably stays within a relatively small range, among the COD range of the catalyst according to the present invention (exceeding 300 ppm and less than 11,000 ppm). When the catalyst is used in a reaction where the number of moles of oxygen required to react with one mole of a raw material substrate is small, on the other hand, a reduction state is preferably more stable because active sites in the reduction state are less likely to be generated during the reaction. In other words, the COD preferably stays within a relatively large range, among the COD range of the catalyst according to the present invention (exceeding 300 ppm and less than 11,000 ppm).

Here, as an example of a reaction producing an α,β-unsaturated aldehyde and/or an α,β-unsaturated carboxylic acid from an alkene, alcohol or ether, a reaction producing methacrolein by oxidizing isobutylene is shown in formula (4) below. Moreover, as an example of a reaction producing an α,β-unsaturated carboxylic acid from an α,β-unsaturated aldehyde, a reaction producing methacrylic acid by oxidizing methacrolein is shown in formula (5) below.

C₄H₈+O₂→C₄H₆O+H₂O (4)

C₄H₆O+_{0.5}O₂→C₄H₆O₂ (5)

The reaction shown in formula (4) above requires 1 mole of oxygen molecules to oxidize 1 mole of a raw material substrate. In contrast, the reaction shown in formula (5) above requires 0.5 moles of oxygen molecules to oxidize 1 mole of the raw material substrate, which is the less amount of moles of oxygen molecules required, as compared to the reaction shown in formula (4) above.

Therefore, when the catalyst according to the present invention is a catalyst used when an α,β-unsaturated aldehyde and/or an α,β-unsaturated carboxylic acid are/is produced from an alkene, alcohol or ether, the COD preferably stays within as a relatively small range. In other words, the COD of the catalyst is preferably exceeding 300 ppm and 2,000 ppm or less. The lower limit of COD of the catalyst is more preferably 400 ppm or more, more preferably 450 ppm or more, particularly preferably 500 ppm or more, and most preferably 550 ppm or more. The upper limit of COD of the catalyst is also preferably 1,500 ppm or less, more preferably 1,400 ppm or less, particularly preferably 1,300 ppm or less, and most preferably 1,200 ppm or less.

Further, when the catalyst according to the present invention is a catalyst used when an α,β-unsaturated carboxylic acid is produced from an α,β-unsaturated aldehyde, the COD preferably stays within a relatively large range. In other words, the COD of the catalyst is preferably 2,500 ppm or more and less than 11,000 ppm. The lower limit of COD of the catalyst is more preferably 2,600 ppm or more, and further preferably 2,700 ppm or more. The upper limit of COD is more preferably 10,000 ppm or less, further preferably 9,000 ppm or less, particularly preferably 8,000 ppm or less, and most preferably 7,500 ppm or less.

Incidentally, the COD of the catalyst in the present invention is measured by the following procedures from (1) to (9).
(1): A triangular flask is charged with 0.2±0.05 g of catalyst which was weighed accurately. The accurately weighed value at this time is m (g).
(2) The triangular flask in (1) is charged with 100 mL of pure water.
(3) The triangular flask in (2) is charged with 10 mL of a sulfuric acid aqueous solution prepared by mixing concentrated sulfuric acid and pure water at a volume ratio of concentrated sulfuric acid:pure water=1:2, and 10 mL of a 5 mmol/L potassium permanganate aqueous solution.
(4): The triangular flask in (3) is immersed in boiling water for 30 minutes.
(5): The liquid in the triangular flask obtained in (4) is filtered through a filter with a mesh aperture of 0.45 μm.
(6): The filtrate obtained in (5) is immersed in boiling water for 5 minutes.
(7): A triangular flask is charged with the filtrate obtained in (6) after the immersion and further added with 10 mL of a 12.5 mmol/L sodium oxalate aqueous solution.
(8): The solution obtained in (7) is titrated with a 5 mmol/L potassium permanganate aqueous solution until it turns light red. The titration volume of the 5 mmol/L potassium permanganate aqueous solution at this time is a (mL).
(9): A COD of the catalyst is calculated based on the m of the catalyst weighed accurately and the titration volume a (mL) of a 5 mmol/L potassium permanganate aqueous solution by using the following formula (3):

$$COD ( ppm ) = 5 \times {10}^{- 3} \times \frac{a}{1000} \times \frac{{10}^{6}}{m} \times 32 \times \frac{5}{4} \qquad ( 3 )$$

wherein in formula (3), the value 5.0×10⁻³ denotes the concentration (mol/L) of potassium permanganate aqueous solution, the value 32 denotes the molecular weight of oxygen molecules, and the value 5/4 denotes (the number of electrons that one molecule of potassium permanganate can oxidize)/(the number of electrons that one molecule of oxygen can oxidize).

Methods of controlling the COD of the catalyst to the above range include, for example, a method of adjusting a composition of the catalyst as described above, or a method of adjusting a type of raw material, a stirring time, a heating time, a heating temperature, calcination conditions, and the like, in the method of producing a catalyst described below. When a composition of the catalyst is prepared, a COD increases by increasing a molar ratio of a transition metal element such as Fe or Cu. In addition, use of methods including step (ii) and step (iii) in the method of producing a catalyst described below allows a catalyst with a specified COD to be easily produced.

(COD/S of Catalyst)

The COD/S of the catalyst represents a weight of oxygen molecules required to completely oxidize the catalyst per unit surface area and is considered to be an index of an abundance ratio of a reduction state of the catalyst on its surface.

The catalyst according to the present invention preferably has a COD/S (μg/m²), which is obtained by dividing a COD of the catalyst by the specific surface area S (m²/g) of the catalyst, of greater than 43 μg/m² and 3600 μg/m² or less. This allows a target product to be produced in higher yield. The reason therefore is conjectured because both an oxidation state and a reduction state of the catalyst are stable on its surface as well, where a catalytic reaction mainly takes place, which thereby facilitates turning of the oxidation-reduction cycle of the catalyst.

In a case in which the catalyst according to the present invention is a catalyst used when an α,β-unsaturated aldehyde and/or an α,β-unsaturated carboxylic acid are/is produced from an alkene, alcohol, or ether, the COD/S of the catalyst is preferably from 45 to 500 μg/m². The lower limit of COD/S of the catalyst is preferably 50 μg/m² or higher. The upper limit of COD/S of the catalyst is more preferably 400 μg/m² or lower, further preferably 300 μg/m² or less, particularly preferably 200 μg/m² or lower, and most preferably 150 μg/m² or lower.

In a case in which the catalyst of the present invention is a catalyst used when an α,β-unsaturated carboxylic acid is produced from an α,β-unsaturated aldehyde, the COD/S of the catalyst is preferably 100 to 3000 μg/m². The lower limit of COD/S of the catalyst is more preferably 200 μg/m² or more, further preferably 300 μg/m² or more, particularly preferably 400 μg/m² or more, and most preferably 500 μg/m² or more. The upper limit of COD/S of the catalyst is also more preferably 2500 μg/m² or less, further preferably 2000 μg/m² or less, and particularly preferably 1500 μg/m² or less.

Incidentally, the specific surface area S of the catalyst in the present invention shall be a value measured by a nitrogen adsorption method (BET one-point method, equilibrium relative pressure=0.30), using a mixture of 30% by volume nitrogen and 70% by volume helium as a measuring gas for 1.0 g of catalyst. The specific surface area can be measured, for example, by using a fully automatic specific surface area analyzer, a Macsorb HM model-1200 (product name, manufactured by MOUNTECH Co., Ltd.).

The specific surface area S of the catalyst can be adjusted, for example, by a calcination temperature and a calcination time in step (v) described below. The specific surface area S tends to become smaller when the calcination temperature is higher and the calcination time is longer. Moreover, when a catalyst having the composition represented by formula (2) is produced, raising a temperature of liquid A3 in step (i-4) described below tends to reduce the specific surface area S.

(Structure of Catalyst)

When the catalyst according to the present invention has the elemental composition represented by formula (2) above, the catalyst preferably contains a Keggin-type heteropolyacid salt from the viewpoint of a target product yield. Note, however, whether or not the catalyst contains a Keggin-type heteropolyacid salt can be confirmed by infrared absorption analysis. The infrared absorption analysis can be carried out by using, for example, a NICOLET 6700 FT-IR (product name, manufactured by THERMO ELECTRON Co., Ltd.). A catalyst containing a heteropolyacid salt having a Keggin-type structure, has characteristic peaks obtained in the vicinity of 1060, 960, 870, and 780 cm⁻¹.

Methods of obtaining a catalyst containing a heteropolyacid salt having a Keggin-type structure, include a method of producing a catalyst by methods including, for example, step (i-3) and (i-4) described below, and setting a pH of liquid A to 4 or lower in step (i-4) above, or a method of calcinating a catalyst at 200° C. or higher in step (v) described below.

[Production Method of Catalyst]

Another embodiment of the present invention is a method of producing a catalyst, which is a method of producing a catalyst containing at least molybdenum, wherein the method includes the following steps (i) to (v). The obtained catalyst preferably has a COD greater than 300 ppm and less than 11,000 ppm.
(i) A step of mixing at least a molybdenum raw material with a solvent to obtain a slurry (liquid A).
(ii) A step of stirring the liquid A at a temperature of 1 to lower than the boiling point of the solvent for 20 to 90 minutes to obtain a slurry (liquid B).
(iii) A step of stirring the liquid B at a temperature of 2° C. or higher than the temperature in step (ii) for 10 minutes to hours to obtain a slurry (liquid C).
(iv) A step of drying the liquid C to obtain a dried product.
(v) A step of calcining the dried product to obtain a catalyst.

Moreover, the method of producing the catalyst according to the present invention may further employ a forming step, which will described below.

Each step will be described in detail below.

(Step (i))

In step (i), at least a molybdenum raw material is mixed with a solvent to obtain a slurry (liquid A). The liquid A is prepared by mixing at least the molybdenum raw material with a solvent. Raw materials of each element contained in formula (1) or (2) above (hereinafter also referred to as catalyst raw material) may be further mixed. The amount of catalyst material used is appropriately adjusted so as to achieve a desired catalyst composition.

The catalyst raw materials are not particularly limited, and each element of nitrates, carbonates, hydrogencarbonates, acetates, ammonium salts, sulfates, oxides, hydroxides, halides, oxoacids, oxoacid salts, and the like may be used singly, or in combinations of two or more types thereof. The COD and COD/S tend to be smaller when compounds that act as oxidizing agents are used as catalyst raw materials, and the COD and COD/S tend to be larger when compounds that act as reducing agents are used as catalyst raw materials.

Examples of the molybdenum raw materials include ammonium paramolybdate, molybdenum trioxide, molybdic acid, molybdenum chloride, and the like, with the ammonium paramolybdate or molybdenum trioxide being preferably used. Examples of bismuth raw materials include bismuth nitrate, bismuth oxide, bismuth subcarbonate, and the like with the bismuth oxide being preferably used. Examples of iron raw materials include iron nitrate, iron hydroxide, iron oxide, and the like, with the iron nitrate being preferably used. Examples of phosphorus raw materials include phosphoric acid, phosphorus pentoxide, ammonium phosphate, cesium phosphate, and the like, with phosphoric acid being preferred. Examples of vanadium raw materials include ammonium metavanadate, vanadium pentoxide, vanadium chloride, and the like, with ammonium metavanadate or vanadium pentoxide being preferably used. Examples of copper raw materials include copper sulfate, copper nitrate, copper oxide, copper carbonate, copper acetate, copper chloride, and the like, and copper nitrate is preferably used. Examples of ammonium root raw materials include ammonium hydrogen carbonate, ammonium carbonate, ammonium nitrate, ammonia water, and the like.

Moreover, raw materials for molybdenum, phosphorus, and vanadium, which are heteropoly acids containing at least one element among molybdenum, phosphorus, and vanadium, may be used. Examples of heteropoly acids include phosphorus molybdic acid, phosphorus vanadomolybdic acid, silico molybdic acid, and the like. These may be used singly or in combinations of two or more thereof.

A solvent is not particularly limited as long as it can dissolve or disperse catalyst materials, but it preferably contains at least water, more preferably it contains water in an amount of 50% by mass or more of the total solvent, further preferably it contains water in an amount of 80% by mass or more of the total solvent, and water may be used singly. The solvent may also contain organic solvents other than water. Examples of organic solvents include but not particularly limited thereto, alcohols, acetone, and the like. The amount of solvent used is not particularly limited, but is preferably to 400 parts by mass relative to 100 parts by mass of the total catalyst materials.

<In the Case of Producing a Catalyst Having the Composition Represented by Formula (1)>

In the case of producing a catalyst having the composition represented by formula (1), step (i) preferably includes the following steps (i-1) and (i-2).

(i-1) A step of preparing a solution or slurry (liquid A1) containing molybdenum, bismuth, and the X and Y elements in formula (1) above, as well as a solution or slurry (liquid A2) containing iron and the M element in formula (1) above.

(i-2) A step of mixing the liquid A1 and liquid A2 to prepare liquid A.

Each step will be described in detail below.

<<Step (i-1)>>

In step (i-1), a solution or slurry (liquid A1) containing molybdenum, bismuth, and the X and Y elements in formula (1) above, and a solution or slurry (liquid A2) containing iron and the M element in formula (1) above, are prepared. Incidentally, the order of preparation of the liquid A1 and liquid A2 is not limited, and the liquid A1 and liquid A2 may be prepared simultaneously.

The amount of each catalyst raw material used is preferably adjusted so that the resulting each catalyst has the composition represented by formula (1) above.

The amount of solvent used is not particularly limited, but that of the liquid A1 is preferably 70 to 400 parts by mass relative to 100 parts by mass of the total catalyst raw materials. The amount of solvent in liquid A2 is preferably to 230 parts by mass relative to 100 parts by mass of the catalyst materials.

<<Step (i-2)>>

In step (i-2), the liquid A1 and liquid A2 obtained in step (i-1) above are mixed to prepare liquid A.

<In the Case of Producing a Catalyst Having the Composition Represented by Formula (2)>

In the case of producing a catalyst having the composition represented by formula (2), the following steps (i-3) and (i-4) are preferably included.

(i-3) A step of preparing a solution or slurry (liquid A3) containing at least molybdenum and phosphorus.

(i-4) A step of preparing liquid A by mixing the liquid A3 with a raw material of the G element in formula (2) above.

Each step will be described below.

<<Step (i-3)>>

In step (i-3), a solution or slurry (liquid A3) containing at least molybdenum and phosphorus is prepared. The liquid A3 preferably contains an element other than the G element in formula (2) above.

The liquid A3 may contain ammonium roots, however, a molar ratio of the ammonium root contained in liquid A3 is preferably 3 or less when a molar ratio of molybdenum in a catalyst to be produced is 12. This results in stable formation of a heteropoly acid structure suitable for production of α,β-unsaturated carboxylic acids in step (i-4) described below. The molar ratio of ammonium root contained in liquid A3 is more preferably 1.5 or less, further preferably 1 or less, and particularly preferably 0.6 or less.

The amount of each catalyst material used is preferably adjusted so that the resulting catalyst has the composition represented by formula (2) above.

The amount of solvent used is not particularly limited, but is preferably 30 to 400 parts by mass relative to 100 parts by mass of the total catalyst materials.

The liquid A3 is preferably prepared by heating to 80 to 130° C. The heating temperature of liquid A3 at 80° C. or higher can sufficiently accelerate a dissolution rate of the catalyst materials. The heating temperature of liquid A3 at 130° C. or lower also inhibits solvent evaporation. The lower limit of the heating temperature of liquid A3 is more preferably 90° C. or higher.

<<Step (i-4)>>

In step (i-4), the liquid A3 obtained in step (i-3) above is mixed with the raw material of the G element in formula (2) above to prepare liquid A. In addition to the raw material of the G element, a raw material of the ammonium root is preferably mixed. This stably forms a heteropoly acid structure suitable for production of an α,β-unsaturated carboxylic acid.

The raw material of G element and the raw material of ammonium root are preferably dissolved or suspended in a solvent to mix them with the liquid A3, and are more preferably dissolved in a solvent to mix with the liquid A3.

In mixing the liquid A3 and the raw material of the G element, a temperature of liquid A3 is preferably from 30 to 99° C. This can inhibit local heat generation of the catalyst when a target product is produced by using the resulting catalyst. The lower limit of the temperature of the A3 liquid is preferably 40° C. or higher, and the upper limit thereof is preferably 95° C. or lower.

The liquid A obtained in step (i-4) preferably contains a Keggin-type heteropolyacid salt from the viewpoint of yields of α,β-unsaturated carboxylic acids. The Keggin-type heteropolyacid salt can be stably formed by allowing a pH of liquid A to be 4 or lower and preferably 2 or lower. Examples of methods of allowing a pH of liquid A to be 4 or lower include a method of appropriately selecting types and the amount of catalyst materials in step (i-3) above and adding nitric acid, oxalic acid, and the like as appropriate to adjust a pH of the liquid A. A pH can be measured with a pH meter. A pH meter, for example, a D-21 (product name, manufactured by HORIBA, Ltd.) can be used.

(Step (ii))

In steps (ii), the liquid A obtained in step (i) is stirred for 20 to 90 minutes at a temperature of 1 to 30° C. lower than the boiling point of the solvent to obtain a slurry (liquid B). For example, in the case of having used water as the solvent in step (i) above, the liquid A is stirred at 70 to 99° C. in step (ii) because the boiling point of water is 100° C. Note, however, in a case in which a plurality of solvents with different boiling points is used in step (i) above, it is stirred at a temperature of 1 to 30° C. lower than a boiling point of a solvent with the largest mass fraction.

In step (ii), solubility of catalyst raw materials to a solvent is adjusted to a constant level by setting a temperature and stirring time to the conditions described above. This is considered to form an active site in which both an oxidation state and a reduction state thereof are stabilized upon formation of an active site of the catalyst in step (iii) described below, which results in providing a catalyst with a COD greater than 300 ppm and less than 11,000 ppm. The temperature in step (ii) being lower than specified or the stirring time being shorter than specified lowers the solubility of the catalyst material, and therefore a COD of the obtained catalyst tends to be 11,000 ppm or more. The temperature in step (ii) being higher than specified or the stirring time being longer than specified, on the contrary, increases the solubility of the catalyst material, and the COD of the obtained catalyst tends to be 300 ppm or less.

The upper limit of temperature upon stirring liquid A is preferably 3° C. or higher below the boiling point of a solvent, and more preferably 5° C. or higher. The lower limit is also preferably 25° C. or lower below the boiling point of the solvent, more preferably 20° C. or lower, and further preferably 10° C. or lower.

The lower limit of the time for stirring at the aforementioned temperature range is preferably 30 minutes or longer and more preferably 40 minutes or longer. The upper limit is preferably 80 minutes or shorter and more preferably minutes or shorter.

(Step (iii))

In step (iii), the liquid B obtained in step (ii) above is stirred for 10 minutes to 10 hours at a temperature of 2° C. or higher than the temperature in step (ii) to obtain a slurry (liquid C).

In step (iii), an active site of the catalyst is formed. In this case, it is considered that stirring the liquid B in which the solubility of the catalyst raw material was adjusted in step (ii), at the temperature described above for the time described above, allows the active site where both the oxidation state and reduction state are stabilized to be formed, thus making it possible to provide a catalyst with a COD greater than 300 ppm and less than 11,000 ppm. The temperature in step (iii) being lower than specified or the stirring time being shorter than specified, renders the reduction state more stable, thereby resulting in providing a likelihood of a COD of the obtained catalyst of 11,000 ppm or more. The temperature in step (iii) being higher than specified or the stirring time being longer than specified, on the other hand, renders the oxidation state stable, thereby providing a likelihood of a COD of the obtained catalyst of 300 ppm or less.

The lower limit of temperature at which the liquid B is stirred is preferably 5° C. or higher than the temperature in step (ii), more preferably 6° C. or higher, and further preferably 8° C. or higher. The upper limit is also preferably or lower above the temperature of step (ii), more preferably 20° C. or lower, and further preferably 10° C. or lower.

Moreover, a temperature at which the liquid B is stirred is preferably a temperature of 1 to 20° C. higher than the boiling point of the solvent. For example, when water is used as the solvent in step (i) above, the liquid B is preferably stirred at 101 to 120° C. in step (iii) because the boiling point of water is 100° C. The lower limit of temperature at which the liquid B is stirred is more preferably 2° C. or higher than the boiling point of the solvent, and more preferably 3° C. or higher. The upper limit is also more preferably 10° C. or lower above the boiling point of the solvent, and further preferably 5° C. or lower.

The lower limit of time for stirring in the temperature range described above is preferably 20 minutes or longer, more preferably 30 minutes or longer, further preferably 60 minutes or longer, particularly preferably 90 minutes or longer, and most preferably 2 hours or longer. The upper limit is also preferably 9 hours or shorter and more preferably 8 hours or shorter.

(Step (iv))

In step (iv), the liquid C obtained in step (iii) above is dried to obtain a dried product.

For drying the liquid C, publicly known methods such as drum drying, air flow drying, evaporation solidification, and spray drying, can be employed. A drying temperature is preferably 120 to 500° C., with the lower limit of 140° C. or higher and the upper limit of 350° C. or lower being more preferred. Drying is preferably carried out so that the moisture content of the resulting dried product is 0.1 to 4.5% by mass. Note, however, these conditions can be appropriately selected depending on a shape and size of a desired catalyst. Implementing drying of liquid C enables inhibition of adhesion of a dried product and improvement of yield.

The dried product obtained in step (iv) may be used as is for calcination in step (v), however, forming improves performance as a catalyst, which is preferred. It is noted that the forming may be carried out after step (v) described below.

(Step (v))

In step (v), the dried product obtained in step (iv) above is calcined to obtain a catalyst. The calcination can also be carried out after the forming step described below has been performed to then obtain a formed product. In the present invention, catalysts including those after calcination and after forming are collectively referred to as catalysts of the present invention.

The calcination may be carried out only once, or it may be divided into a plurality of times together with the forming step described below. For example, primary calcination may be carried out first, the forming step described below may be carried out for the resulting primarily calcinated product, and secondary calcination may be carried out for the resulting formed product. Moreover, the primary calcination and secondary calcination may be carried out, and a forming step may be carried out for the resulting catalyst. Moreover, the forming step described below is carried out first, and then the resulting formed product may be calcinated.

The calcination can be carried out under gas flow distribution of oxygen-containing gases such as air, an inert gas, or a reducing gas. The term “inert gas” refers to a gas that does not lower catalytic activity, such as nitrogen, carbon dioxide, helium, or argon. Examples of reducing gases include hydrogen, a propylene gas, an isobutylene gas, an acrolein gas, a methacrolein gas, and the like. These may be used singly or in combinations of two or more thereof. Calcination under the gas flow distribution of oxygen-containing gases such as air, tends to reduce a COD and COD/S of the catalyst, while calcination under the gas flow distribution of an inert gas or a reducing gas tends to increase the COD and COD/S of the catalyst.

A calcination temperature is preferably 200 to 700° C. The lower limit of the calcination temperature is more preferably 300° C. or higher, while the upper limit is more preferably 500° C. or lower and further preferably 450° C. or lower.

A calcination time is preferably 0.5 to 40 hours, while the lower limit is preferably 1 hour or longer. The calcination temperature raised and the calcination time prolonged tend to increase a COD/S, while the calcination temperature lowered and the calcination time shortened tend to decrease a COD/S. It is noted that the calcination time refers to a time required to hold a predetermined calcination temperature after it was reached.

Of these described above, when the catalyst is a catalyst used when an α,β-unsaturated aldehyde and/or an α,β-unsaturated carboxylic acid are/is produced from an alkene, alcohol or ether, or a catalyst having the composition represented by formula (1) above, a dried product preferably undergoes primary calcination followed by forming, and the resulting formed product preferably undergoes secondary calcination.

In this case, a calcination temperature of the primary calcination is preferably 200 to 600° C., with the lower limit of 250° C. or higher and the upper limit of 450° C. or lower being more preferred. A calcination time for the primary calcination is preferably 0.5 to 5 hours from the viewpoint of improving target product yields. A type of calcination furnace and calcination methods upon the primary calcination are not particularly limited, and for example, a box-type calcination furnace, a tunnel furnace type calcination furnace or the like may be used to calcinate a dried product or a formed product in a fixed condition. Moreover, a rotary kiln and the like may be used to calcinate the dried product or formed product while it is flowed.

The calcination temperature of the secondary calcination is preferably from 300 to 700° C., with the lower limit of 400° C. or higher and the upper limit of 600° C. or lower being more preferred. A calcination time of the secondary calcination is preferably 10 minutes to 10 hours from the viewpoint of improving target product yields, with the lower limit of 1 hour or longer being more preferred. A type of calcination furnace and calcination methods upon the secondary calcination are not particularly limited, and for example, a box-type calcination furnace, a tunnel furnace type calcination furnace or the like may be used to calcinate a formed product or a primary calcinated product in a fixed condition. Moreover, a rotary kiln and the like may be used to calcinate the dried product or primary calcinated product while it is flowed.

Moreover, when the catalyst is a catalyst used when an α,β-unsaturated carboxylic acid is produced from an α,β-unsaturated aldehyde or a catalyst having the composition represented by formula (2) above, forming is preferably carried out on a dried product, and the resulting formed product is preferably calcinated.

(Forming Step)

In the forming step, the dried product obtained in step (iv) above or the calcinated product obtained in step (v) above is formed to obtain a formed product. The forming methods are not particularly limited, and publicly known dry or wet forming methods can be employed. Examples thereof include tableting forming, extrusion forming, pressure forming, rolling granulation, and the like.

Upon forming, publicly known additives, for example, organic compounds, such as a polyvinyl alcohol and a carboxymethylcellulose, may be added. Furthermore, inorganic compounds such as graphite, talc and silicon soil, and inorganic fibers such as glass fibers, ceramic fibers, and carbon fibers may be added.

A shape of the formed product is not particularly limited and can be arbitrary shapes, such as spherical, cylindrical, ring, star shape, or a granular shape of a formed product that was crushed and classified after forming, and the like. Of these, preferable are spherical, cylindrical, and ring shapes from the viewpoint of a mechanical strength. A size of the formed product is not particularly limited, but it is preferably, 0.1 to 10 mm of a diameter of sphere, for example, in the case of a spherical shape. The lower limit of the diameter of the sphere is preferably 0.5 mm or larger, more preferably 1 mm or larger, and particularly preferably 3 mm or larger. The upper limit of the diameter of the sphere is also more preferably 8 mm or smaller, and further preferably 6 mm or smaller. In the case of a ring shape or cylinder shape, a diameter of a circle at the bottom of the ring or cylinder and height of the ring or cylinder are both preferably to 10 mm. The lower limit of the diameter and height are more preferably 0.5 mm or larger, further preferably 1 mm or larger, and particularly preferably 3 mm or larger. The upper limit of the diameter and height are also more preferably 8 mm or smaller and further preferably 6 mm or smaller. For other shapes, a length between the two most distant points in a solid body of a catalyst is preferably 0.1 to 10 mm. The lower limit of the length between two points is more preferably mm or more, further preferably 1 mm or more, and particularly preferably 3 mm or more. The upper limit of the length between the two points is also more preferably 8 mm or less, and further preferably 6 mm or less. This improves target product yields and a catalyst life.

An outer surface area of a formed product is not particularly limited, but from the viewpoint of stable production of a target product over a long period of time, the lower limit thereof is preferably 0.01 cm² or more, more preferably 0.05 cm² or more, and further preferably 0.1 cm² or more. From the viewpoint of improving target product yields, the upper limit is, on the other hand, preferably 4 cm² or less, more preferably 3 cm² or less, and further preferably 2 cm² or less.

The volume of formed product is not particularly limited, but from the viewpoint of stable production of the target product over a long period of time, the lower limit thereof is preferably 0.0001 cm³ or more, more preferably 0.001 cm³ or more, and further preferably 0.01 cm³ or more. From the viewpoint of improving yields of target products, the upper limit is, on the other hand, preferably 5 cm³ or less, more preferably 1 cm³ or less, and further preferably 0.5 cm³ or less.

The mass of formed product is not particularly limited, but from the viewpoint of stable production of the target product over a long period of time, the lower limit thereof is preferably 0.002 g/product or more, more preferably 0.01 g/product or more, and further preferably 0.05 g/product or more. From the viewpoint of improving target product yields, the upper limit is, on the other hand, preferably 0.5 g/product or less, more preferably 0.3 g/product or less, and further preferably 0.2 g/product or less.

The filling bulk density of formed product is not particularly limited, but from the viewpoint of stable production of the target product over a long period of time, the lower limit thereof is preferably 0.2 g/cm³ or higher, more preferably 0.3 g/cm³ or higher, and further preferably g/cm³ or higher. From the viewpoint of improving the target product yields, the upper limit is, on the other hand, preferably 2 g/cm³ or lower, more preferably 1.5 g/cm³ or lower, further preferably 1.3 g/cm³ or lower, and particularly preferably 0.8 g/cm³ or lower. Note that the filling bulk density of a formed product shall refer to a value calculated from the total mass of a formed product upon being filled into a 100 ml graduated cylinder by the method in accordance with JIS-K 7365.

The resulting formed product may be supported on a support. Examples of supports used upon supporting include silica, alumina, silica-alumina, magnesia, titania, silicon carbide, and the like. The formed product can also be diluted with inert materials such as silica, alumina, silica-alumina, magnesia, titania, and silicon carbide and used.

The catalyst can be produced in such a manner as described above.

[Method of Producing α,β-Unsaturated Aldehyde and/or α,β-Unsaturated Carboxylic Acid]

In the method of producing α,β-unsaturated aldehydes and/or α,β-unsaturated carboxylic acids according to the present invention, the catalyst according to the present invention or a catalyst produced by the production method according to the present invention is used to produce the corresponding α,β-unsaturated aldehydes and/or α,β-unsaturated carboxylic acids from an alkene, alcohol, or ether.

In the method of producing an α,β-unsaturated aldehyde and/or an α,β-unsaturated carboxylic acid according to the present invention, a catalyst having the composition represented by formula (1) above is preferably used, and a catalyst with a COD of greater than 300 ppm and 2,000 ppm or less is preferably used.

Examples of the aforementioned alkenes include propylene, isobutylene, and the like. Examples of the alcohols also include t-butyl alcohol, isobutyl alcohol, and the like. Examples of the ethers also include methyl-t-butyl ether and the like. Oxidation of these raw organic compounds enables production of the corresponding α,β-unsaturated aldehydes and/or α,β-unsaturated carboxylic acids. For example, when the raw organic compound is propylene, the corresponding α,β-unsaturated aldehyde is acrolein, and the corresponding α,β-unsaturated carboxylic acid is acrylic acid. Moreover, in a case in which the raw organic compound is isobutylene, t-butyl alcohol, isobutyl alcohol, or methyl-t-butyl ether, the corresponding α,β-unsaturated aldehyde is methacrolein, and the corresponding α,β-unsaturated carboxylic acid is methacrylic acid. From the viewpoint of yields of target products, an α,β-unsaturated aldehyde and an α,β-unsaturated carboxylic acid are preferably (meth)acrolein and (meth)acrylic acid, respectively, with methacrolein and methacrylic acid being more preferred. Note that “(meth)acrolein” denotes acrolein and methacrolein, and “(meth)acrylic acid” denotes acrylic acid and methacrylic acid.

The method of producing an α,β-unsaturated aldehyde and/or an α,β-unsaturated carboxylic acid, according to the present invention can be carried out by contacting the catalyst according to the present invention or a catalyst produced by the production method according to the present invention, and a raw material gas containing the raw organic compound and oxygen in a reactor.

The reactor is not particularly limited, but a tube reactor equipped with reaction tubes filled with a catalyst is preferably used, and industrially a multi-tube reactor equipped with a plurality of reaction tubes is particularly preferably used. A catalyst layer inside the reactor may be a single catalyst layer, or a plurality of catalysts with different activity may be each separated and filled to a plurality of layers. The catalyst may also be diluted with an inert support to control the activity and then filled.

A concentration of the raw organic compound in the raw material gas is preferably 1 to 20% by volume, with the lower limit of 3% by volume or more and the upper limit of 10% by volume or less being more preferred. Note, however, the raw organic compound may contain a small amount of impurities such as a lower saturated alkane that does not substantially affect the present reaction.

A concentration of oxygen in the raw material gas is preferably 0.1 to 5 moles relative to 1 mole of raw organic compound, with the lower limit of 0.5 moles or more and the upper limit of 3 moles or less being more preferred. Air is preferred as an oxygen source for the raw material gas from an economic point of view. Gas enriched with oxygen by mixing pure oxygen with air or the like may also be used, if necessary.

The raw material gas may be diluted with an inert gas such as nitrogen or carbon dioxide gas for the economic point of view. Furthermore, water vapor may be added to the raw material gas. A reaction in the presence of water vapor allows an α,β-unsaturated aldehyde and/or an α,β-unsaturated carboxylic acid to be obtained in higher yield. A concentration of water vapor in the raw material gas is preferably 0.1 to 50% by volume, with the lower limit of 1% by volume or more and the upper limit of 40% by volume or less being more preferred.

A reaction pressure is preferably 0 to 1 MPa (G). Here, “(G)” is a gauge pressure, and 0 MPa (G) means that the reaction pressure is an atmospheric pressure. A reaction temperature is also preferably 200 to 450° C., with the lower limit of 250° C. or higher and the upper limit of 400° C. or lower being more preferred.

A contact time between the raw material gas and the catalyst is preferably 0.5 to 15 seconds. The lower limit of the contact time is more preferably 1 second or longer, while the upper limit is more preferably 10 seconds or shorter, and further preferably 5 seconds or shorter.

The production in such a manner described above allows α,β-unsaturated aldehydes and/or α,β-unsaturated carboxylic acids corresponding to the raw organic compounds used to be obtained in high yield.

[Method of Producing α,β-Unsaturated Carboxylic Acid]

In the method of producing an α,β-unsaturated carboxylic acid according to the present invention, the catalyst of the present invention or a catalyst produced by the production method of the present invention is used to produce from an α,β-unsaturated aldehyde, the corresponding α,β-unsaturated carboxylic acid. Incidentally, the α,β-unsaturated aldehyde may be produced by the method of producing an α,β-unsaturated aldehyde and/or an α,β-unsaturated carboxylic acid according to the present invention.

In the method of producing an α,β-unsaturated carboxylic acid, a catalyst having the composition represented by formula (2) above is preferably used and a catalyst with COD of greater than 2,500 ppm and less than 11,000 ppm is also preferably used.

When an α,β-unsaturated carboxylic acid is produced from an α,β-unsaturated aldehyde produced by the production method according to the present invention, the catalyst according to the present invention or a catalyst produced by the production method according to the present invention may also be used, or any other publicly known catalyst may be used.

Examples of the α,β-unsaturated aldehydes include (meth)acrolein, crotonaldehyde (β-methyl acrolein), cinnamaldehyde (β-phenyl acrolein), and the like. An α,β-unsaturated carboxylic acid to be produced is an α,β-unsaturated carboxylic acid in which an aldehyde group of the aforementioned α,β-unsaturated aldehyde was changed to a carboxyl group. Specifically, when the α,β-unsaturated aldehyde is (meth)acrolein, (meth)acrylic acid is obtained. From the viewpoint of target product yields, the α,β-unsaturated aldehyde and the α,β-unsaturated carboxylic acid are preferably (meth)acrolein and (meth)acrylic acid, respectively and more preferably methacrolein and methacrylic acid.

The method of producing an α,β-unsaturated carboxylic acid according to the present invention can be carried out by contacting the catalyst according to the present invention or a catalyst produced by the production method according to the present invention, and a raw material gas containing an α,β-unsaturated aldehyde and oxygen in a reactor. The reactor that is the same as that in the production method of the α,β-unsaturated aldehyde and/or the α,β-unsaturated carboxylic acid described above, can be used. A catalyst layer inside the reactor may be a single catalyst layer, or a plurality of catalysts with different activity may be each separated and filled to a plurality of layers. The catalyst may also be diluted with an inert support to control the activity and then filled.

A concentration of α,β-unsaturated aldehyde in the raw material gas is preferably 1 to 20% by volume, with the lower limit of 3% by volume or more, and the upper limit of 10% by volume or less being more preferred. Note, however, the α,β-unsaturated aldehyde may contain a small amount of impurities such as a lower saturated aldehyde that does not substantially affect the present reaction.

A concentration of oxygen in the raw material gas is preferably 0.4 to 4 moles relative to 1 mole of α,β-unsaturated aldehyde, with the lower limit of 0.5 moles or more and the upper limit of 3 moles or less being more preferred. Air is preferred as an oxygen source for the raw material gas from an economic point of view. Gas enriched with oxygen by mixing pure oxygen with air or the like may be used, if necessary.

From an economic standpoint, the raw material gas may be diluted with an inert gas such as nitrogen or a carbon dioxide gas. Furthermore, water vapor may be added to the raw material gas. A reaction in the presence of water vapor enables an α,β-unsaturated carboxylic acid to be obtained in higher yield. A concentration of water vapor in the raw material gas is preferably 0.1 to 50% by volume, with the lower limit of 1% by volume or more and the upper limit of 40% by volume or less being more preferred.

A reaction pressure is preferably 0 to 1 MPa (G). A reaction temperature is also preferably 200 to 450° C., with the lower limit of 250° C. or higher and the upper limit of 400° C. or lower being preferred.

A contact time between the raw material gas and the catalyst is preferably 0.5 to 15 seconds. The lower limit is more preferably 1 second or longer, while the upper limit is more preferably 10 seconds or shorter and further preferably seconds or shorter.

[Production Method of α,β-Unsaturated Carboxylic Acid Ester]

In the method of producing an α,β-unsaturated carboxylic acid ester according to the present invention, an α,β-unsaturated carboxylic acid produced by the production method according to the present invention, is esterified. Alcohols to be reacted with an α,β-unsaturated carboxylic acid are not particularly limited, and examples of the alcohols include methanol, ethanol, propanol, isopropanol, butanol, isobutanol, and the like. Examples of the resulting α,β-unsaturated carboxylic acid esters include, for example, methyl (meth)acrylate, ethyl (meth)acrylate, propyl (meth)acrylate, isopropyl (meth)acrylate, butyl (meth)acrylate, isobutyl (meth)acrylate, and the like. The reaction can be carried out in the presence of acidic catalysts such as a sulfonic acid type cation exchange resin. A reaction temperature is preferably 50 to 200° C.

### EXAMPLES

Hereinafter, the present invention will be described in detail by way of Examples and Comparative Examples, however, the present invention is not limited to these Examples. Note, however, the “parts” in Examples and Comparative Examples refer to parts by mass.

(Composition of Catalyst)

A molar ratio of each element in the catalyst was determined by analyzing components of the catalyst dissolved in ammonia water by ICP atomic emission spectrometry. An ICP Optima 8300 (manufactured by Perkin Elmer Inc.) was used as an analyzer, with an output of 1300 W, plasma gas flow rate: 10 L/min, auxiliary gas flow rate: 0.2 L/min, nebulizer gas flow rate: 0.55 L/min, and detector: split array type CCD.

Moreover, a molar ratio of an ammonium root was determined by analyzing the catalyst by the Kjeldahl method.

(COD of Catalyst)

The COD of the catalyst is measured by the following procedures from (1) to (9).
(1): A triangular flask is charged with 0.2±0.05 g of catalyst which was weighed accurately. The accurately weighed value at this time is m (g).
(2) The triangular flask in (1) is charged with 100 mL of pure water.
(3) The triangular flask in (2) is added with 10 mL of a sulfuric acid aqueous solution prepared by mixing concentrated sulfuric acid and pure water at a volume ratio of concentrated sulfuric acid:pure water=1:2, and 10 mL of a 5 mmol/L potassium permanganate aqueous solution.
(4): The triangular flask in (3) is immersed in boiling water for 30 minutes.
(5): The liquid in the triangular flask obtained in (4) is filtered through a filter with a mesh aperture of 0.45 μm.
(6): The filtrate obtained in (5) is immersed in boiling water for 5 minutes.
(7): A triangular flask is charged with the filtrate obtained in (6) after the immersion and further added with 10 mL of a 12.5 mmol/L sodium oxalate aqueous solution.
(8): The solution obtained in (7) is titrated with a 5 mmol/L potassium permanganate aqueous solution until it turns light red. The titration volume of the 5 mmol/L potassium permanganate aqueous solution is a (mL).
(9): A COD of the catalyst is calculated based on the m of the catalyst weighed accurately and the titration volume a (mL) of the 5 mmol/L potassium permanganate aqueous solution by using formula (3) above.

(Specific Surface Area of Catalyst)

The specific surface area S of the catalyst is measured by employing the nitrogen adsorption method (BET one-point method, equilibrium relative pressure=0.30) and using a mixture of 30% by volume nitrogen and 70% by volume helium as a measuring gas for 1.0 g of catalyst. The specific surface area can be measured, for example, using a fully automatic specific surface area analyzer, a Macsorb HM model-1200 (product name, manufactured by MOUNTECH Co., Ltd.).

(Reaction Evaluation)

Reactions of the catalysts in Examples 1 to 3 and Comparative Examples 1 and 2 were evaluated by taking production of methacrolein and methacrylic acid by oxidation of isobutylene as an example. The raw material gases and products in the reaction evaluation were analyzed by using the following gas chromatography. A piece of apparatus and columns used are listed below.

Apparatus: A GC-2014 manufactured by Shimadzu Corporation

Column (methacrolein): A 007-CW-60W-3.0F (length: 60 m, inner diameter: 0.32 mm, and film thickness: 3.0 μm) manufactured by QUADREX Corporation

Column (methacrylic acid): A DB-FFAP (length: 30 m, inner diameter: 0.32 mm, and film thickness: 1.0 μm) manufactured by J&W Corporation

From the results of gas chromatography, the total yield of methacrolein and methacrylic acid was determined by the following equation.

Total yield of methacrolein and methacrylic acid (%)=(P1+P2)/F1×100

In the above formula, F1 is the number of moles of isobutylene supplied per unit time, P1 is the number of moles of methacrolein formed per unit time, and P2 is the number of moles of methacrylic acid formed per unit time.

Further, reactions of the catalysts in Examples 4 to 6 and Comparative Examples 3 and 6 were evaluated by taking production of methacrylic acid by oxidation of methacrolein as an example. The raw material gases and formed products in the reaction evaluation were analyzed by using the same gas chromatography as described above. From the results of gas chromatography, the yield of methacrylic acid was determined by the following equation.

Yield of methacrylic acid (%)=P2/F2×100

In the above formula, F2 is the number of moles of methacrolein supplied per unit time, and P2 is the number of moles of methacrylic acid formed per unit time.

### Example 1

Liquid A1 was obtained by mixing 500 parts by mass of ammonium paramolybdate tetrahydrate, 12.3 parts by mass of ammonium para tungstate, 27.6 parts by mass of cesium nitrate, 38.5 parts by mass of bismuth (III) oxide, and 20.6 parts by mass of antimony trioxide with 2,000 parts by mass of pure water at 60° C. as a solvent. Separately from the liquid A1, liquid A2 was also obtained by mixing 200.2 parts by mass of iron (III) nitrate nonahydrate and 515.1 parts by mass of cobalt (II) nitrate hexahydrate in 1,000 parts by mass of pure water. The liquid A1 and liquid A2 were then mixed to obtain liquid A.

The obtained liquid A was heated to 95° C. and stirred for 1 hour while maintaining the liquid temperature at 95° C. to obtain liquid B.

The resulting liquid B was heated to 103° C. and stirred for 7 hours while maintaining the liquid temperature at 103° C. to obtain liquid C.

The resulting liquid C was dried in a spray dryer to obtain a dried product. The dried product did not adhere to the wall of the spray dryer and was in favorable dry condition. A composition of the dried product, excluding oxygen, was Mo₁₂Bi_{0.7}Fe_{2.1}Co_{7.5}W_{0.2}Sb_{0.6}Cs_{0.6}(NH₄)_{10.5}.

The resulting dried product underwent primary calcination at 300° C. for 1 hour under an air atmosphere, and the dried product after calcination was then pressure-formed followed by pulverized to obtain pulverized particles. Thereafter the pulverized particles were subjected to secondary calcination at 500° C. for 6 hours in an air atmosphere to obtain a catalyst.

The obtained catalyst underwent measurements of COD and specific surface area S. The calculated COD and COD/S values are shown in Table 1.

Thereafter, the obtained catalyst was filled in a reaction tube to form a catalyst layer, and an oxidation reaction of isobutylene was carried out under the following conditions. The results are shown in Table 1.

Composition of a raw material gas: 5% by volume of isobutylene, 12% by volume of oxygen, 10% by volume of water vapor, and 73% by volume of nitrogen

Reaction temperature: 340° C.

Contact time between raw material gas and catalyst: 3 seconds.

### Example 2

A dried product was obtained by the same method as in Example 1. The dried product did not adhere to the wall of the spray dryer and was in favorable dry condition. A composition of the dried product, excluding oxygen, was Mo₁₂Bi_{0.7}Fe_{2.1}Co_{7.5}W_{0.2}Sb_{0.6}Cs_{0.6}(NH₄)_{10.5}.

The resulting dried product underwent primary calcination by the same method as in Example 1. The dried product after calcination then underwent extrusion forming to obtain a ring-shaped formed product with an outer diameter of 5 mm, an inner diameter of 2 mm, and a length of 5.5 mm. The formed product then was subjected to secondary calcination at 500° C. for 6 hours under an air atmosphere to obtain a catalyst.

The obtained catalyst underwent measurements of COD and specific surface area S. The calculated COD and COD/S values are shown in Table 1.

Thereafter, the obtained catalyst was filled in a reaction tube to form a catalyst layer, and an oxidation reaction of isobutylene was carried out by the same method as in Example 1. The results are shown in Table 1.

### Example 3

Liquid B was obtained by the same method as in Example 1.

The obtained liquid B was heated to 103° C. and stirred for 3 hours while maintaining the temperature at 103° C. to obtain liquid C.

The resulting liquid C was dried in a spray dryer to obtain a dried product. The dried product did not adhere to the wall of the spray dryer and was in favorable dry condition. A composition of the dried product, excluding oxygen, was Mo₁₂Bi_{0.7}Fe_{2.1}Co_{7.5}W_{0.2}Sb_{0.6}Cs_{0.6}(NH₄)_{10.5}.

The resulting dried product underwent primary calcination, forming, and secondary calcination by the same method as in Example 2 to obtain a catalyst.

The obtained catalyst underwent measurements of COD and specific surface area S. The calculated COD and COD/S values are shown in Table 1.

Thereafter, the obtained catalyst was filled in a reaction tube to form a catalyst layer, and an oxidation reaction of isobutylene was carried out by the same method as in Example 1. The results are shown in Table 1.

### Comparative Example 1

Liquid B was obtained by the same method as in Example 1.

The obtained liquid B was dried in a spray dryer to obtain a dried product. In other words, step (iii) was not carried out, and the liquid B was dried to obtain the dried product. The dried product did not adhere to the wall of the spray dryer and was in favorable dry condition. A composition of the dried product, excluding oxygen, was Mo₁₂Bi_{0.7}Fe_{2.1}Co_{7.5}W_{0.2}Sb_{0.6}Cs_{0.6}(NH₄)_{10.5}.

The resulting dried product underwent primary calcination, forming, and secondary calcination by the same method as in Example 1 to obtain a catalyst.

The obtained catalyst underwent measurements of COD and specific surface area S. The calculated COD and COD/S values are shown in Table 1.

Thereafter, the obtained catalyst was filled in a reaction tube to form a catalyst layer, and an oxidation reaction of isobutylene was carried out by the same method as in Example 1. The results are shown in Table 1.

### Comparative Example 2

Liquid A was obtained by the same method as in Example 1.

The obtained liquid A was heated to 95° C. and stirred for 2 hours while maintaining the liquid temperature at 95° C. to obtain liquid B′. In other words, the liquid B′ was obtained by having stirred it for a longer time than 90 minutes in step (ii).

The obtained liquid B′ was heated to 100° C. and stirred for 1 hour while maintaining the liquid temperature at 100° C. to obtain liquid C.

The resulting liquid C was evaporated to dryness to obtain a dried product. A composition of the dried product, excluding oxygen, was Mo₁₂Bi_{0.7}Fe_{2.1}Co_{7.5}W_{0.2}Sb_{0.6}Cs_{0.6}(NH₄)_{10.5}.

The resulting dried product underwent primary calcination, forming, and secondary calcination to obtain a catalyst.

The obtained catalyst underwent measurements of COD and specific surface area S. The calculated COD and COD/S values are shown in Table 1.

Thereafter, the obtained catalyst was filled in a reaction tube to form a catalyst layer, and an oxidation reaction of isobutylene was carried out by the same method as in Example 1. The results are shown in Table 1.

TABLE 1

| | | | | | | | Total yield of |
|---|---|---|---|---|---|---|---|
| | Step (ii) | | Step (iii) | | | | methacrolein and |
| | Temperature | Time | Temperature | Time | COD | COD/S | methacrylic acid |
| | (° C.) | (hr) | (° C.) | (hr) | (ppm) | (μg/m2) | (%) |
| Example 1 | 95 | 1 | 103 | 7 | 1000 | 91 | 86.1 |
| Example 2 | 95 | 1 | 103 | 7 | 590 | 54 | 85.5 |
| Example 3 | 95 | 1 | 103 | 3 | 1100 | 100 | 85.6 |
| Comparative | 95 | 1 | — | — | 300 | 43 | 73.8 |
| Example 1 | | | | | | | |
| Comparative | 95 | 2 | 100 | 1 | 300 | 27 | 80.1 |
| Example 2 | | | | | | | |

### Example 4

To 2,000 parts by mass of pure water at 25° C. as a solvent were added 500 parts by mass of molybdenum trioxide, 17 parts by mass of ammonium metavanadate, a solution in which 47 parts by mass of an 85% by mass phosphoric acid aqueous solution was diluted in 30 parts by mass of pure water, and a solution in which 10.5 parts by mass of copper (II) nitrate trihydrate was dissolved in 22.5 parts by mass of pure water. The resulting slurry was heated to 95° C. under stirring, and stirred for 2 hours while maintaining the liquid temperature at 95° C. to obtain liquid A3. Thereafter, while maintaining the temperature of the liquid A3 at 95° C., a solution of 56 parts by mass of cesium hydrogen carbonate dissolved in 100 parts by mass of pure water and a solution of 46 parts by mass of ammonium carbonate dissolved in 132 parts by mass of pure water were mixed under stirring to obtain liquid A.

The obtained liquid A was heated to 95° C. and stirred for 20 minutes to obtain liquid B.

The resulting liquid B was heated to 98° C. and stirred for 15 minutes while maintaining the liquid temperature at 98° C. to obtain liquid C.

The resulting liquid C was dried in a spray dryer to obtain a dried product. A composition of the dried product, excluding oxygen, was P_{1.4}Mo₁₂V_{0.5}Cu_{0.15}Cs₁(NH₄)_{3.3}.

The resulting dried product underwent extrusion forming to form a cylindrical shape with a diameter of 5.5 mm and a height of 5.5 mm, and calcined at 380° C. for 10 hours under an air atmosphere to obtain a catalyst.

The obtained catalyst contained a Keggin-type heteropolyacid salt. The obtained catalyst also underwent measurements of COD and specific surface area S. The calculated COD and COD/S values are shown in Table 2.

Thereafter, the obtained catalyst was filled in a reaction tube to form a catalyst layer, and an oxidation reaction of methacrolein was carried out under the following conditions. The results are shown in Table 2.

Composition of raw material gas: 5% by volume of methacrolein, 10% by volume of oxygen, 30% by volume of water vapor, and 55% by volume of nitrogen

Reaction temperature: 300° C.

Contact time between raw material gas and catalyst: 2 seconds

### Example 5

To 1,000 parts by mass of pure water at 25° C. as a solvent were added 500 parts by mass of molybdenum trioxide, 20.5 parts by mass of ammonium metavanadate, 36.5 parts by mass of an 85% by mass phosphoric acid aqueous solution, a solution in which 7 parts by mass of copper (II) nitrate trihydrate was dissolved in 61 parts by mass of pure water, and a solution in which 6 parts by mass of Iron(III) nitrate nonahydrate was diluted in 25 parts by mass of pure water. The resulting slurry was heated to 95° C. under stirring, and stirred for 2 hours while maintaining the liquid temperature at 95° C. to obtain liquid A3. Thereafter, the liquid A3 was lowered from a temperature of 95° C. to 50° C., and a solution of 73 parts by mass of cesium nitrate dissolved in 125 parts pure water and 199 parts by mass of a 25% by mass ammonia water were mixed, while the liquid temperature was kept at 50° C. under stirring, to then obtain liquid A.

The obtained liquid A was heated to 70° C. and stirred for 20 minutes while maintaining the liquid temperature at 70° C. to obtain liquid B.

The resulting liquid B was heated to 101° C. and stirred for 2 hours while maintaining the liquid temperature at 101° C. to obtain liquid C.

The resulting liquid C was dried in a drum dryer to obtain a dried product. A composition of the dried product, excluding oxygen, was P_{1.1}Mo₁₂V_{0.6}Cu_{0.1}Fe_{0.05}Cs_{1.3}(NH₄)_{10.7}.

The resulting dried product underwent tableting forming to form a cylindrical shape with a diameter of 5.5 mm and a height of 5.5 mm, and calcined at 380° C. for 10 hours under an air atmosphere to obtain a catalyst.

The obtained catalyst contained a Keggin-type heteropolyacid salt. The obtained catalyst also underwent measurements of COD and specific surface area S. The calculated COD and COD/S values are shown in Table 2.

Thereafter, the obtained catalyst was filled in a reaction tube to form a catalyst layer, and an oxidation reaction of methacrolein was carried out by the same method as in Example 4. The results are shown in Table 2.

### Example 6

A dried product was obtained by the same method as in Example 5. The dried product did not adhere to the wall of the spray dryer and was in favorable dry condition. A composition of the dried product, excluding oxygen, was P_{1.1}Mo₁₂V_{0.6}Cu_{0.1}Fe_{0.05}Cs_{1.3}(NH₄)_{10.7}.

The resulting dried product underwent tableting forming to form a cylindrical shape with a diameter of 5.5 mm and a height of 5.5 mm, and underwent primary calcination at 380° C. for 10 hours under an air atmosphere followed by secondary calcination at 305° C. for 2 hours under a methacrolein gas atmosphere to obtain a catalyst.

The obtained catalyst contained a Keggin-type heteropolyacid salt. The obtained catalyst also underwent measurements of COD and specific surface area S. The calculated COD and COD/S values are shown in Table 2.

Thereafter, the obtained catalyst was filled in a reaction tube to form a catalyst layer, and an oxidation reaction of methacrolein was carried out by the same method as in Example 4. The results are shown in Table 2.

### Comparative Example 3

Liquid B was obtained by the same method as in Example 4.

The obtained liquid B was dried in a spray dryer to obtain a dried product. In other words, step (iii) was not carried out, and the liquid B was dried to obtain the dried product. A composition of the dried product, excluding oxygen, was P_{1.4}Mo₁₂V_{0.5}Cu_{0.15}Cs_{1.3}(NH₄)_{3.3}.

The resulting dried product underwent extrusion forming to form a cylindrical shape with a diameter of 5.5 mm and a height of 5.5 mm, and underwent primary calcination at 380° C. for 10 hours under an air atmosphere followed by secondary calcination at 301° C. for 16 hours under a methacrolein gas atmosphere to obtain a catalyst.

The obtained catalyst contained a Keggin-type heteropolyacid salt. The obtained catalyst also underwent measurements of COD and specific surface area S. The calculated COD and COD/S values are shown in Table 2.

Thereafter, the obtained catalyst was filled in a reaction tube to form a catalyst layer, and an oxidation reaction of methacrolein was carried out by the same method as in Example 4. The results are shown in Table 2.

### Comparative Example 4

A reagent, phosphomolybdic acid (manufactured by NIPPON INORGANIC COLOUR & CHEMICAL CO., LTD.) was used as a dried product. A composition of the dried product, excluding oxygen, was P₁Mo₁₂.

The dried product underwent pressure forming, and the resulting formed product was pulverized and calcined at 300° C. for 5 hours under an air atmosphere to obtain a catalyst.

The obtained catalyst contained a Keggin-type heteropolyacid salt. The obtained catalyst also underwent measurements of COD and specific surface area S. The calculated COD and COD/S values are shown in Table 2.

Thereafter, the obtained catalyst was filled in a reaction tube to form a catalyst layer, and an oxidation reaction of methacrolein was carried out by the same method as in Example 4. The results are shown in Table 2.

### Comparative Example 5

A reagent, phospho vanadomolybdic acid (manufactured by NIPPON INORGANIC COLOUR & CHEMICAL CO., LTD.) was used as a dried product. A composition of the dried product, excluding oxygen, was P_{1.1}Mo₁₂V_{1.1}.

The dried product underwent forming and calcination by the same method as in Comparative Example 3 to obtain a catalyst.

The obtained catalyst contained a Keggin-type heteropolyacid salt. The obtained catalyst also underwent measurements of COD and specific surface area S. The calculated COD and COD/S values are shown in Table 2.

Thereafter, the obtained catalyst was filled in a reaction tube to form a catalyst layer, and an oxidation reaction of methacrolein was carried out by the same method as in Example 4. The results are shown in Table 2.

### Comparative Example 6

To 800 parts by mass of pure water at 25° C. as a solvent were added 16.9 parts by mass of molybdenum trioxide, 1.0 parts by mass of vanadium pentoxide, and 1.2 parts by mass of an 85% by mass phosphoric acid aqueous solution. The resulting slurry was heated to 85° C. with stirring, and stirred for 3 hours while maintaining the liquid temperature at 85° C. to obtain liquid A.

The obtained liquid A was heated to 90° C. and stirred for 1 hour while maintaining the liquid temperature at 90° C. to obtain liquid B.

The resulting liquid B was evaporated to dryness to obtain a dried product. In other words, step (iii) was not carried out, and the liquid B was dried to obtain the dried product. A composition of the dried product, excluding oxygen, was P_{1.1}Mo₁₂V_{1.1}.

The resulting dried product was formed and calcinated by the same method as in Comparative Example 4 to obtain a catalyst.

The obtained catalyst contained a Keggin-type heteropolyacid salt. The obtained catalyst also underwent measurements of COD and specific surface area S. The calculated COD and COD/S values are shown in Table 2.

Thereafter, the obtained catalyst was filled in a reaction tube to form a catalyst layer, and an oxidation reaction of methacrolein was carried out by the same method as in Example 4. The results are shown in Table 2.

TABLE 2

| | Step (ii) | | Step (iii) | | | | Total yield of |
|---|---|---|---|---|---|---|---|
| | Temperature | Time | Temperature | Time | COD | COD/S | methacrylic acid |
| | (° C.) | (min) | (° C.) | (min) | (ppm) | (μg/m2) | (%) |
| Example 4 | 70 | 20 | 98 | 15 | 7309 | 2436 | 45.9 |
| Example 5 | 70 | 20 | 101 | 120 | 2700 | 540 | 35.9 |
| Example 6 | 70 | 20 | 101 | 120 | 6000 | 1200 | 38.3 |
| Comparative | 70 | 20 | — | — | 11000 | 3667 | 26.9 |
| Example 3 | | | | | | | |
| Comparative | Use of reagent phosphomolybdic acid | | | | 73 | 24 | 18.8 |
| Example 4 | | | | | | | |
| Comparative | Use of reagent phospho vanadomolybdic acid | | | | 45 | 6 | 11.4 |
| Example 5 | | | | | | | |
| Comparative | 90 | 60 | — | — | 170 | 24 | 18.0 |
| Example 6 | | | | | | | |

As shown in Table 1, the total yield of methacrolein and methacrylic acid was favorable in Examples 1 to 3, where the COD was within the specified range.

Incidentally, methacrylic acid can be obtained by oxidizing the methacrolein obtained in Examples, and a methacrylic acid ester can be obtained by esterifying the methacrylic acid.

As also shown in Table 2, Examples 4 to 6, where the COD was within the specified range, each demonstrated the favorable yield of methacrylic acid.

Note, however, the methacrylic acid obtained in Examples can be esterified to obtain a methacrylic acid ester.

### INDUSTRIAL APPLICABILITY

According to the present invention, a catalyst capable of producing target products such as an α,β-unsaturated aldehyde and/or an α,β-unsaturated carboxylic acid in high yield, can be provided, which is industrially useful.

What is claimed is:

1. A catalyst comprising at least molybdenum, wherein a COD (chemical oxygen demand) of the catalyst is greater than 300 ppm and less than 11,000 ppm.

2. The catalyst according to claim 1, wherein a value obtained by dividing the COD value (ppm) by a value of specific surface area S (m²/g) of the catalyst (COD/S) is greater than 43 μg/m² and 3600 μg/m² or less.

3. The catalyst according to claim 1, for use in production of an α,β-unsaturated aldehyde and/or an α,β-unsaturated carboxylic acid from an alkene, an alcohol, or an ether.

4. The catalyst according to claim 1, having a composition represented by the following formula (1):
Moₐ₁Bi_{b1}Fe_{c1}M_{d1}Xₑ₁Y_{f1}Si_{g1}(NH₄)ₕ₁Oᵢ₁ (1)
wherein in formula (1) above, Mo, Bi, Fe, Si, NH₄, and O each represent molybdenum, bismuth, iron, silicon, an ammonium root, and oxygen; M represents at least one element selected from the group consisting of cobalt and nickel; X represents at least one element selected from the group consisting of zinc, chromium, lead, manganese, calcium, magnesium, niobium, silver, barium, tin, tantalum, tungsten, antimony, phosphorus, boron, sulfur, selenium, tellurium, cerium and titanium; Y represents at least one element selected from the group consisting of lithium, sodium, potassium, rubidium, cesium, and thallium; a1, b1, c1, d1, e1, f1, g1, h1, and it each represent a molar ratio of each component, and when a1=12, b1=0.01 to 3, c1=0 to 8, d1=0 to 12, e1=0 to 8, f1=0.001 to 2, g1=0 to 20, h1=0 to 30, and it is a molar ratio of oxygen required to satisfy a valence of the each component.

5. The catalyst according to claim 3, wherein the COD is greater than 300 ppm and 2,000 ppm or less.

6. The catalyst according to claim 3, wherein the COD is from 400 to 1,500 ppm.

7. The catalyst according to claim 3, wherein a value obtained by dividing the COD value (ppm) by a value of specific surface area S (m²/g) of the catalyst (COD/S) is from 45 to 500 μg/m².

8. The catalyst according to claim 1, for use in production of an α,β-unsaturated carboxylic acid from an α,β-unsaturated aldehyde.

9. The catalyst according to claim 1, having a composition represented by the following formula (2):
Pₐ₂Mo_{b2}V_{c2}Cu_{d2}Aₑ₂E_{f2}G_{g2}(NH₄)ₕ₂Oᵢ₂ (2)
wherein in formula (2) above, P, Mo, V, Cu, NH₄, and O each represent phosphorus, molybdenum, vanadium, copper, an ammonium root, and oxygen; A represents at least one element selected from the group consisting of antimony, bismuth, arsenic, germanium, zirconium, tellurium, silver, selenium, silicon, tungsten and boron; E represents at least one element selected from the group consisting of iron, zinc, chromium, magnesium, calcium, strontium, tantalum, cobalt, nickel, manganese, barium, titanium, tin, lead, niobium, indium, sulfur, palladium, gallium, cerium, and lanthanum; G represents at least one element selected from the group consisting of lithium, sodium, rubidium, potassium, cesium and thallium; a2, b2, c2, d2, e2, f2, g2, h2 and i2 each represent a molar ratio of each component, and when b2=12, a2=0.5 to 3, c2=0.01 to 3, d2=0.01 to 2, e2=0 to 3, f2=0 to 3, g2=0 to 5, h2=0 to 30, and i2 is a molar ratio of oxygen required to satisfy a valence of the each component.

10. The catalyst according to claim 8, wherein the COD is 2,500 ppm or greater and less than 11,000 ppm.

11. The catalyst according to claim 8, wherein the COD is from 2,600 to 10,000 ppm.

12. The catalyst according to claim 8, wherein a value obtained by dividing the COD value (ppm) by a value of specific surface area S (m²/g) of the catalyst (COD/S) is from 100 to 3,000 μg/m².

13. A method of producing a catalyst comprising at least molybdenum, wherein the method comprises the following steps (i) to (v):
(i) mixing at least a molybdenum raw material with a solvent to obtain a slurry (liquid A);
(ii) stirring the liquid A at a temperature of 1 to 30° C. lower than the boiling point of the solvent for 20 to 90 minutes to obtain a slurry (liquid B);
(iii) stirring the liquid B at a temperature of 2° C. or higher than the temperature in the step (ii) for 10 minutes to 10 hours to obtain a slurry (liquid C);
(iv) drying the liquid C to obtain a dried product; and
(v) calcining the dried product to obtain a catalyst.

14. The method of producing the catalyst according to claim 13, wherein 50% by mass or more of the total solvent is water in the step (i).

15. The method of producing the catalyst according to claim 13, wherein the temperature in the step (iii) is 1 to 20° C. higher than the boiling point of the solvent.

16. The method of producing a catalyst according to claim 13, comprising stirring the liquid B for 90 minutes to 10 hours to obtain the liquid C in the step (iii).

17. The method of producing a catalyst according to claim 13, comprising calcining the dried product under oxygen-containing gas distribution in the step (v).

18. The method of producing a catalyst according to claim 13, comprising producing a catalyst used when an α,β-unsaturated aldehyde and/or an α,β-unsaturated carboxylic acid are/is produced from an alkene, an alcohol, or an ether.

19. The method of producing a catalyst according to claim 13, comprising producing a catalyst used when an α,β-unsaturated carboxylic acid is produced from an α,β-unsaturated aldehyde.

20. A method of producing an α,β-unsaturated aldehyde and/or an α,β-unsaturated carboxylic acid, comprising producing the α,β-unsaturated aldehyde and/or the α,β-unsaturated carboxylic acid from an alkene, an alcohol or an ether by using the catalyst according to claim 1.

21. A method of producing an α,β-unsaturated aldehyde and/or an α,β-unsaturated carboxylic acid, comprising producing the α,β-unsaturated aldehyde and/or the α,β-unsaturated carboxylic acid from an alkene, an alcohol or an ether by using a catalyst produced by the production method according to claim 13.

22. A method of producing an α,β-unsaturated carboxylic acid, comprising producing the α,β-unsaturated carboxylic acid from an α,β-unsaturated aldehyde by using the catalyst according to claim 1.

23. A method of producing an α,β-unsaturated carboxylic acid, comprising producing the α,β-unsaturated carboxylic acid from an α,β-unsaturated aldehyde by using a catalyst produced by the production method according to claim 13.

24. A method of producing an α,β-unsaturated carboxylic acid, comprising producing the α,β-unsaturated carboxylic acid from an α,β-unsaturated aldehyde produced by the production method according to claim 20.

25. A method of producing an α,β-unsaturated carboxylic acid ester, comprising producing the α,β-unsaturated carboxylic acid ester from an α,β-unsaturated carboxylic acid produced by the production method according to claim 20.
